(19) United States
(12) Patent Application Publication
(10) Pub. No.: US 2025/0025420 A1
(43) Pub. Date: Jan. 23, 2025

(54) TERNARY CO-AMORPHOUS FORMS OF DRUGS, PROTEINS, AND WATER-SOLUBLE POLYMERS

(71) Applicant: Zerion Pharma APS, Birkerød (DK)

(72) Inventors: Korbinian Löbmann, Copenhagen N (DK); Donglei Leng, Søborg (DK); Wei Tian, Cleghorn (GB); Bulut Bulduk, Taastrup (DK); Ole Wiborg, Birkerød (DK)

(21) Appl. No.: 18/713,118

(22) PCT Filed: Nov. 24, 2022
(86) PCT No.: PCT/EP2022/083157

§ 371 Date: May 23, 2024

(30) Foreign Application Priority Data
Nov. 24, 2021 (EP) 21210275.0

(51) Int. Cl. A61K 9/14

(52) U.S. Cl. CPC A61K 9/146

(57) ABSTRACT

The present invention concerns ternary co-amorphous forms including a drug compound, a nutraceutical, or a dietary supplement in combination with a protein and a water-soluble polymer. Co-amorphous forms are a combination of three or more components that form a homogeneous amorphous system where the components are intimately mixed on the molecular level. The ternary co-amorphous forms of the invention provide increased solubility and dissolution rate of drug compounds, nutraceuticals, and dietary supplements. This in turn increases the bioavailability of these compounds.

### FIELD OF THE INVENTION

The present invention concerns ternary co-amorphous forms including a drug compound, a nutraceutical, or a dietary supplement in combination with a protein and a water-soluble polymer. Such ternary co-amorphous forms provide increased solubility and dissolution rate of the drug compound, nutraceutical, and dietary supplement. This in turn increases the bioavailability of these compounds.

### BACKGROUND OF THE INVENTION

It is known in the art that the solubility and dissolution of an otherwise poorly soluble drug, nutraceutical or dietary supplement molecules can be increased by forming a co-amorphous form of these molecules with a water-soluble polymer.

More recently, the co-amorphous forms of an API and a protein have been shown to increase the solubility and dissolution rate of the API (WO 2018/113890 and WO 2021/110983) to a greater extent than water-soluble polymers. This increase is more or less pronounced depending on the API in question.

Surprisingly, it has now been found that solubility and dissolution rate can be increased even more by preparing ternary co-amorphous forms of a drug, a nutraceutical or a dietary supplement compound together with a protein and a water-soluble polymer.

### SUMMARY OF THE INVENTION

In one aspect, the present invention concerns a co-amorphous form of a drug, a nutraceutical, or a dietary supplement with a protein and a water-soluble polymer.

These ternary co-amorphous forms have been found to provide unexpectedly high solubilities of compounds having very low aqueous solubility compared to binary co-amorphous forms comprising either a protein or a water-soluble polymer.

In another aspect, the present invention concerns a method of preparing the co-amorphous form according to the invention, wherein the drug, nutraceutical, or dietary supplement, protein, and water-soluble polymer together are subjected to spray drying, solvent evaporation, freeze drying, precipitation from supercritical fluids, melt quenching, hot melt extrusion, electrospinning, 2D printing, 3D printing, and any milling process, such as wet milling, ball milling and cryo-milling.

In a further aspect, the present invention concerns the use of a protein and a water-soluble polymer for preparing a co-amorphous form with a drug, nutraceutical, or dietary supplement.

### BRIEF DESCRIPTION OF THE FIGURES

FIG. 1: XRPD diffractograms of binary and ternary co-amorphous forms of Compound A at 25% (w/w) drug loading (a), Compound B at 23% (w/w) drug loading (b), binary and ternary formulations of Abiraterone acetate at 40% and 56% (w/w) drug loadings (c), binary and ternary formulations of Ibrutinib at 50% (w/w) drug loading (d), binary formulations of Cannabidiol BSPG at 20% and 30% (w/w) drug loadings and ternary formulations of Cannabidiol BSPG at 30% (w/w) drug loading (e).

FIG. 2: Powder dissolution of binary and ternary co-amorphous forms of Compound A at 25% (w/w) drug loading in FaSSGF (a) and FaSSIF-V2 (b).

FIG. 3: Powder dissolution of binary and ternary co-amorphous forms of Compound B at 23% (w/w) drug loading in FaSSIF-V1.

FIG. 4: Powder dissolution of binary and ternary co-amorphous forms of Abiraterone acetate at 40% and 56% (w/w) drug loadings in FaSSIF-V1.

FIG. 5: Powder dissolution of binary and ternary co-amorphous forms of Ibrutinib at 50% (w/w) drug loading in FaSSIF-V2.

FIG. 6: Powder dissolution of crystalline Cannabidiol BSPG, binary co-amorphous form of Cannabidiol BSPG at 20% and 30% (w/w) drug loadings, and ternary co-amorphous forms of Cannabidiol BSPG at 30% (w/w) drug loading in FaSSIF-V2.

### DETAILED DESCRIPTION OF THE INVENTION

### Definitions

In the context of the present invention, the term “co-amorphous” refers to a combination of three or more components that form a homogeneous amorphous system where the components are intimately mixed in a single phase or two phases. X-ray powder diffraction (XRPD), together with Differential Scanning calorimetry (DSC), can be used to identify whether the sample is “co-amorphous” after preparation, e.g. by measuring the absence of Bragg peaks and the appearance of one or two glass transition temperature(s), i.e. less than the at least three Tg's that would be observed if no mixing on the molecular level took place. Typically, “co-amorphous form” refers to a form with a single Tg.

In the context of the present invention, the term “purity” in connection with a protein in a co-amorphous form, such as beta-lactoglobulin, is defined as a percentage (w/w) of the total amount of protein comprised in the co-amorphous form. When the co-amorphous form is comprised in a pharmaceutical composition, any additional protein, such as gelatin, that may be included as an excipient in the pharmaceutical formulation does not enter into the calculation of the purity of the beta-lactoglobulin comprised in the co-amorphous form. Furthermore, if an additional protein is included as an excipient in a pharmaceutical composition, said additional protein may give rise to an additional glass transition temperature (if amorphous) or melting point (if crystalline) in addition to the glass transition temperature of the co-amorphous form.

In the context of the present invention, the term “nutraceutical” means a food, or parts of a food, not registered or requiring registration as a drug or medicinal product, and which may provide medical or health benefits, including the prevention and treatment of disease. The term “dietary supplement” is defined by the FDA as a vitamin; mineral; herb or other botanical; amino acid; dietary substance for use by man to supplement the diet by increasing the total dietary intake; or a concentrate, metabolite, constituent, extract, or combination of the preceding substances “. Thus, “dietary supplement” constitutes a subset of “nutraceutical”.

In the context of the present invention, the term “drug, nutraceutical or dietary supplement compound” is intended to refer to an active pharmaceutical ingredient, a veterinary medicinal product, or a product containing a dietary ingredient, such as vitamins, minerals, amino acids, and herbs or botanicals, as well as other substances that can be used to supplement the diet. In one embodiment, the term “drug, nutraceutical or dietary supplement compound” refers to an active pharmaceutical ingredient. When referring to “a” drug, nutraceutical or dietary supplement compound in the context of the present invention, it may refer to one or more drug or dietary supplement compounds.

In the context of the present invention, the term “protein” refers to a synthetic, recombinant or natural polypeptide, preferably comprising at least 40 amino acids. The term “protein” also includes modified proteins, such as protein hydrolysates or modifications obtained by phosphorylation, glycosylation, lipidation, ubiquitination, carbonylation, nitrosylation, methylation, acetylation, palmitoylation, amidation, oxidation, nitration, formylation, sulfation, hydroxylation, myristoylation, When referring to “water-soluble polymer” in the context of the present invention, reference is made to a polymer that is not peptide-based, i.e. less than 10% of the monomers used to form the polymer are amino acids, e.g. less than 5%, such as less than 1%, more particularly 0%.

### The (Co-) Amorphous Form(s)

In one aspect, the present invention concerns a co-amorphous form of a drug, a nutraceutical, or a dietary supplement with a protein and a water-soluble polymer. The co-amorphous form of the invention may have one or two phases. In one embodiment, it has a single phase.

The co-amorphous form of the invention may contain from 1 to 99% (w/w) of the drug, nutraceutical, or dietary supplement, such as from 5 to 95% (w/w) of the drug, nutraceutical, or dietary supplement. In one embodiment, the co-amorphous form comprises from 10 to 90% (w/w) of the drug, nutraceutical, or dietary supplement and from 10 to 90% (w/w) of protein and water-soluble polymer. In a further embodiment, the co-amorphous form comprises from 20 to 90% (w/w) of the drug, nutraceutical, or dietary supplement and from 10 to 80% (w/w) of protein and water-soluble polymer. In still a further embodiment, the co-amorphous form comprises from 30 to 85% (w/w) of the drug, nutraceutical, or dietary supplement and from 15 to 70% (w/w) of protein and water-soluble polymer. In another embodiment, the co-amorphous form comprises from 50 to 85% (w/w) of the drug, nutraceutical, or dietary supplement and from 15 to 50% (w/w) of protein and water-soluble polymer. In a further embodiment, the co-amorphous form comprises from 55 to 75% (w/w) of the drug, nutraceutical, or dietary supplement and from 25 to 45% (w/w) of protein and water-soluble polymer. In yet another embodiment, the co-amorphous form comprises 30% (w/w) of the drug, nutraceutical, or dietary supplement and 70% (w/w) of protein and water-soluble polymer. In yet a further embodiment, the co-amorphous form comprises 50% (w/w) of the drug, nutraceutical, or dietary supplement and 50% (w/w) of protein and water-soluble polymer. In still a further embodiment, the co-amorphous form comprises 60% (w/w) of the drug, nutraceutical, or dietary supplement and 40% (w/w) of protein and water-soluble polymer. In yet another embodiment, the co-amorphous form comprises 70% (w/w) of the drug, nutraceutical, or dietary supplement and 30% (w/w) of protein and water-soluble polymer.

It has been found that lower drug loadings provide particularly good dissolution of drug molecules having low solubility, especially for drug molecules having very low solubility. Accordingly, in one embodiment, the co-amorphous form comprises from 5 to 35% (w/w) of the drug, nutraceutical, or dietary supplement and from 65 to 95% (w/w) of protein and water-soluble polymer. In a further embodiment, the co-amorphous form comprises from 10 to 30% (w/w) of the drug, nutraceutical, or dietary supplement and from 70 to 90% (w/w) of protein and water-soluble polymer. In still a further embodiment, the co-amorphous form comprises from 12 to 25% (w/w) of the drug, nutraceutical, or dietary supplement and from 75 to 88% (w/w) of protein and water-soluble polymer. In yet a further embodiment, the co-amorphous form comprises from 15 to 20% (w/w) of the drug, nutraceutical, or dietary supplement and from 80 to 85% (w/w) of protein and water-soluble polymer.

The relative amounts of protein and water-soluble polymer may be adjusted as needed for preparing co-amorphous forms having the desired level of solubility. In one embodiment, the weight ratio between protein and water-soluble polymer is in the range 10:1 to 1:10. In another embodiment, the weight ratio between protein and water-soluble polymer is in the range 5:1 to 1:5.

In yet another embodiment, the weight ratio between protein and water-soluble polymer is in the range 5:1 to 1:3. In still another embodiment, the weight ratio between protein and water-soluble polymer is in the range 4:1 to 1:2. In yet another embodiment, the weight ratio between protein and water-soluble polymer is in the range 3:1 to 2:3. In a further embodiment, the weight ratio between protein and water-soluble polymer is in the range 2:1 to 1:1.

In a further aspect, the present invention concerns the use of a protein and a water-soluble polymer for preparing a co-amorphous form with a drug, nutraceutical, or dietary supplement.

The co-amorphous forms may be prepared according to the present examples or according to the general methods disclosed in WO 2018/113890. Accordingly, in one aspect, the present invention concerns a method of preparing a co-amorphous form of the invention, said method selected from subjecting the drug, nutraceutical, or dietary supplement and beta-lactoglobulin together to spray drying, solvent evaporation, freeze drying, precipitation from supercritical fluids, melt quenching, hot melt extrusion, electrospinning, 2D printing, 3D printing, and any milling process, such as wet milling, ball milling and cryo-milling.

### The Protein

Proteins (peptide-based polymers) have been shown to increase solubility of low-soluble pharmaceutical ingredients (WO 2017/186889, WO 2018/113890 and WO 2021/110983) when prepared with active pharmaceutical ingredients as co-amorphous forms. In one embodiment, the protein is selected from the group consisting of whey protein isolate, soy protein, soy protein hydrolysate, pea protein, corn protein, wheat protein, hemp protein, rye protein, oat protein, peanut protein, barley protein, myoglobin, lysozyme, egg protein isolate, egg white protein isolate, egg white protein hydrolysate, ovalbumin, casein, beta-lactoglobulin, alpha-lactalbumin, lactoferrin, gelatine, albumin, serum albumin, bovine serum albumin, human serum albumin, egg albumin, fish albumin, keratin, elastin, collagen, immunoglobulin G, rice protein isolate, rice protein hydrolysate, yeast protein hydrolysate, and mixtures thereof. In another embodiment, the protein is beta-lactoglobulin.

It has in particular been found that beta-lactoglobulin of high purity contributes to co-amorphous forms having high solubilities. Thus, in one embodiment, the purity of the beta-lactoglobulin is at least 92% (w/w) of the total amount of protein. In a further embodiment of the present invention, the purity of the beta-lactoglobulin is at least 94% (w/w) of the total amount of protein. In another embodiment of the present invention, the purity of the beta-lactoglobulin is at least 95% (w/w) of the total amount of protein. In still another embodiment of the present invention, the purity of the beta-lactoglobulin is at least 96% (w/w) of the total amount of protein. In yet another embodiment, the purity of the beta-lactoglobulin is at least 97% (w/w) of the total amount of protein. In a further embodiment of the present invention, the purity of the beta-lactoglobulin is at least 98% (w/w) of the total amount of protein.

### The Water-Soluble Polymer

A number of polymer excipients are known in the art for increasing the dissolution rate and/or for increasing the aqueous solubility of active ingredients when formulated with active pharmaceutical ingredients in tablets, capsules etc. The water-soluble polymer is soluble in an aqueous medium in at least a portion of the pH range 1 to 14 at 25° C. Thus, the water-soluble polymer includes types of polymers soluble only at some PH levels, e.g. polymers soluble at acidic pH but not at neutral pH. Water-soluble polymers suitable for use with the present invention are known in the art. Water-soluble polymers suitable for use with the present invention may be cellulosic or non-cellulosic. The polymers may be neutral or ionizable in aqueous solution.

A suitable class of water-soluble polymers comprises polymers that are “amphiphilic” in nature, meaning that the polymer has hydrophobic and hydrophilic portions. The hydrophobic portion may comprise groups, such as aliphatic or aromatic hydrocarbon groups. The hydrophilic portion may comprise either ionizable or non-ionizable groups that are capable of hydrogen bonding such as hydroxyls, carboxylic acids, esters, amines or amides.

One class of water-soluble polymers suitable for use with the present invention comprises neutral non-cellulosic polymers. Exemplary polymers include: vinyl polymers and copolymers having substituents of hydroxyl, alkylacyloxy, or cyclicamido; polyvinyl alcohols that have at least a portion of their repeat units in the unhydrolyzed (vinyl acetate) form; polyvinyl alcohol polyvinyl acetate copolymers; polyvinyl pyrrolidone; polyoxyethylene-polyoxypropylene copolymers, also known as poloxamers; and polyethylene polyvinyl alcohol copolymers.

Another class of water-soluble polymers suitable for use with the present invention comprises ionizable non-cellulosic polymers. Exemplary polymers include: carboxylic acid-functionalized vinyl polymers, such as the carboxylic acid functionalized polymethacrylates and carboxylic acid functionalized polyacrylates such as the EUDRAGITS® manufactured by Evonik (Darmstadt, Germany), amine-functionalized polyacrylates and polymethacrylates; and carboxylic acid functionalized starches such as starch glycolate.

Non-cellulosic water-soluble polymers that are amphiphilic are copolymers of a relatively hydrophilic and a relatively hydrophobic monomer. Examples include acrylate and methacrylate copolymers, and polyoxyethylene-polyoxypropylene copolymers. Exemplary commercial grades of such copolymers include the EUDRAGITS, which are copolymers of methacrylates and acrylates, and the PLURONICS supplied by BASF, which are polyoxyethylene-polyoxypropylene copolymers.

A further class of water-soluble polymers suitable for use with the present invention comprises cellulosic polymers, which may be ionizable or neutral. Neutral amphiphilic cellulosic water-soluble polymers comprise polymers in which the parent cellulosic polymer has been substituted at any or all of the 3 hydroxyl groups present on each saccharide repeat unit with at least one relatively hydrophobic substituent. Hydrophobic substituents may be essentially any substituent that, if substituted to a high enough level or degree of substitution, can render the cellulosic polymer essentially aqueous insoluble. Examples of hydrophobic substituents include ether-linked alkyl groups such as methyl, ethyl, propyl, butyl, etc.; or ester-linked alkyl groups such as acetate, propionate, butyrate, etc.; and ether—and/or ester-linked aryl groups such as phenyl, benzoate, or phenylate. Hydrophilic regions of the polymer can be either those portions that are relatively unsubstituted, since the unsubstituted hydroxyls are themselves relatively hydrophilic, or those regions that are substituted with hydrophilic substituents. Hydrophilic substituents include ether—or ester-linked nonionizable groups such as the hydroxy alkyl substituents hydroxyethyl, hydroxypropyl, and the alkyl ether groups such as ethoxyethoxy or methoxyethoxy. Particularly preferred hydrophilic substituents are those that are ether—or ester-linked ionizable groups such as carboxylic acids, thiocarboxylic acids, substituted phenoxy groups, amines, phosphates or sulfonates.

One class of cellulosic water-soluble polymers comprises neutral polymers, meaning that the polymers are substantially non-ionizable in aqueous solution. Such polymers contain non-ionizable substituents, which may be either ether-linked or ester-linked. Exemplary ether-linked non-ionizable substituents include: alkyl groups, such as methyl, ethyl, propyl, butyl, etc.; and hydroxy alkyl groups such as hydroxymethyl, hydroxyethyl, hydroxypropyl, etc. Exemplary ester-linked non-ionizable substituents include: alkyl groups, such as acetate, propionate, butyrate, etc. Exemplary neutral polymers that may be used as the water-soluble polymer include: hydroxypropyl methyl cellulose acetate, hydroxypropyl methyl cellulose, hydroxypropyl cellulose, methyl cellulose, hydroxyethyl methyl cellulose, hydroxyethyl cellulose acetate, and hydroxyethyl ethyl cellulose.

In one embodiment, the water-soluble polymer is selected from PVP (polyvinylpyrrolidone), PVAc (Polyvinyl acetate), PVOH (Polyvinyl alcohol), PVAP (Polyvinyl acetate phthalate), Poloxamer type polymers (e.g. Poloxamer 188), PEG, MC, HPMC, HPC, HEC, HPMCAS, HPMCP, chitosan, polymethacrylate-based copolymers (e.g. Eudragit type polymers), polyacrylic acid polymers (e.g. Carbomer or Carbopol 940), carboxymethyl cellulose acetate butyrate and other cellulose derivatives, modified starches and other polysaccharides, polyvinyl caprolactam-polyvinyl acetate-polyethylene glycol graft co-polymer (Soluplus), and different PVPVA grades (e.g. PVPVA64), as well as any mixture thereof.

In a further embodiment, the water-soluble polymer is selected from polyvinyl pyrrolidone, Poloxamer type polymers, polymethacrylate-based copolymers, polystyrene sulfonate, as well as any mixture thereof. In still a further embodiment, the water-soluble polymer is polystyrene sulfonate.

### The Drug, Nutraceutical or Dietary Supplement Compound

The drug, nutraceutical or dietary supplement compound used in the method according to the invention may in principle be any pharmaceutically active ingredient, nutraceutical or dietary supplement compound suitable for human consumption. Typically, such compounds have low solubility in water. This can in particular be a problem for drug compounds needing reproducibility in terms of bioavailability. Thus, the present invention is particularly useful for drug compounds. Accordingly, in one embodiment, the drug, nutraceutical or dietary supplement compound is a drug compound. In a further embodiment, the drug compound is classified in BCS class II or IV. These classes of drug compounds refer to “low solubility and high permeability” and “low solubility and low permeability”, respectively. In another embodiment, the drug, nutraceutical or dietary supplement compound is selected from the group consisting of abiraterone, abiraterone acetate, aceclofenac, acetazolamide, acetylsalicylic acid, aclidinium bromide, acyclovir, afamelanotide acetate, albendazole, albuterol sulfate, aliskiren fumarate, allopurinol, alprostadil, amantadine hydrochloride, aminolevulinic acid hydrochloride, amiodarone hydrochloride, amoxicillin, amprenavir, anagrelide hydrochloride, anidulafungin, apalutamide, apixaban, apremilast, aprepitant, apriprazole, atorvastatin, azelaic acid, azithromycin, benidipine, bazedoxifene acetate, bedaquiline fumarate, benzonatate, bexarotene, bicalutamide, binimetinib, bisacodyl, brivaracetam, budesonide, candesartan, carbamazepine, cabergoline, cannabidiol, cannabinoids, carfilzomib, carisoprodol, carvedilol, cefdinir, cefditoren, cefixime, cefotiam, cefpodoxime, cefuroxime axetil, celecoxib, chlarithromycin, chloroquine, chlorpromazine, ciclesonide, cilexetil, cilostazol, ciprofloxacin, cladribine, clarithromycin, clofazimine, clonazepam, clopidogrel, clozapine, cobicistat, colistimethate sodium, curcumin, cyclosporine, cyproterone, dabrafenib mesylate, dapaglifozin, dapsone, daptomycin, dasabuvir, dasatinib, deferasirox, delafloxacin meglumine, dexamethasone, dexmethylphenidate hydrochloride, diazepam, diclofenac, diloxanide, docetaxel, dolutegravir sodium, doxycycline, dutasteride, duvelisib, ebastine, efavirenz, eluxadoline, elvitegravir, empagliflozin, enasidenib mesylate, enzalutamide, epalrestat, eprosartan, erythromycin, eslicarbazepine acetate, essential fatty acids, estradiol, estrone sulphate, ethyl icosapentate, etoposide, etravirine, everolimus, ezetimibe, famotidine, fenofibrate, flibanserin, fluocinonide, flurbiprofen, fluticasone furoate, fluticasone propionate, folic acid, formoterol fumarate, furosemide, gefitinib, glatiramer acetate, glibenclamide, gliclazide, glimpiride, glipizide, glycopyrrolate, griseofulvin, haloperidol, hydrochlorothiazide, hydrocortisone, hydroxyzine, ibuprofen, ibrutinib, icosapent ethyl, imatinib, indinavir, irbesartan, irinotecan, isotretinoin, itraconazole, ivacaftor, ivermectin, ketoprofen, L-carbocysteine, lamotrigine, lenalidomide, lesinurad, letermovir, levalbuterol tartrate, levodopa, levonorgestrel, linezolid, lopinavir, loratadine, lorazepam, lovastatin, lubiprostone, manidipine, mebendazole, medroxyprogesterone, mefloquine, megestrol acetate, melatonin, meloxicam, melphalan, menatetrenone, mercaptopurine, mesalamie, metaxalone, methylphenidate, metoclopramide, metoprolol, metronidazole, midostaurin, modafinil, mometasone furoate, morphine sulfate, mosapride, mycamine, nabilone, nabumetone, nalidixic acid, naproxen sodium, nelfinavir, nepafenac, nevirapine, neratinib, nicergoline, niclosamide, nifedipine, nilotinib, nilotinib hydrochloride monohydrate, nilvadipine, nimesulide, nimodipine, nintedanib, nitisinone, nitrofurantoin, norethindrone acetate, nystatin, olanzapine, olaparib, olmesartan, omadacycline, opicapone, orlistat, ospemifene, oxcarbazepine, oxycodone, paclitaxel, paliperidone palmitate, palonosetron hydrochloride, paricalcitol, pazopanib hydrochloride, perampanel, phenobarbital, phenytoin, pioglitazone, pitavastatin, posaconazole, pranlukast, praziquantel, prednisolone acetate, prednisone, progesterone, pyrantel, pyrimethamine, quetiapine, quinine, raloxifene, rebamipide, regorafenib, retinol, ribociclib succinate, rifampicin, rifaximin, rilpivirine, rimegepant, riociguat, risperidone, ritonavir, rivaroxaban, rofecoxib, rolapitant hydrochloride, roxithromycin, rucaparib, safinamide mesylate, saquinavir, sennoside A, sertraline, sevelamer carbonate, sildenafil, simeprevir, simvastatin, sirolimus, sofosbuvir, sonidegib phosphate, sorafenib tosylate, spironolactone, sufentanil citrate, sugammadex sodium, sulfadiazine, sulfamethoxazole, sulfasalazine, sultamicillin, sulpiride, sunitinib malate, suvorexant, tacrolimus, tadalafil, tafamidis, tafamidis meglumine, tamoxifen, tasimelteon, tecovirimat, telaprevir, telmisartan, telotristat ethyl, teprenone, teriflunomide, theophylline, ticlopidine, tipranavir, tocopherol nicotinate, tolterodine tartrate, topotecan hydrochloride, tosufloxacin, tretinoin, triflusal, trimethoprim, umeclidinium bromide, uridine triacetate, ursodeoxycholic acid, valproic acid, valsartan, vandetanib, vemurafenib, venetoclax, vitamin A, vitamin D, verapamil, voriconazole, warfarin, ziprasidone hydrochloride and zaltoprofen.

### Solubility Increase

The co-amorphous form according to the invention increases solubility of the drug, nutraceutical or dietary supplement and does so with an increased solubility compared to binary co-amorphous forms according to the state of the art. Accordingly, in one embodiment, the solubility of the drug, nutraceutical or dietary supplement in the co-amorphous form with protein and water-soluble polymer is increased compared to the solubility of the drug, nutraceutical or dietary supplement in a binary co-amorphous form with the same protein.

In a further embodiment, the solubility of the drug, nutraceutical or dietary supplement in the co-amorphous form with protein and water-soluble polymer is increased compared to the solubility of the drug, nutraceutical or dietary supplement in a binary co-amorphous form with the same water-soluble polymer.

In another embodiment, the increase in solubility compared to binary co-amorphous forms is at least 50%. In still another embodiment, the increase is at least 70%. In yet another embodiment, the increase is at least 100%.

### General

It should be understood that any feature and/or aspect discussed above in connections with the compounds according to the invention apply by analogy to the methods described herein.

The following figures and examples are provided below to illustrate the present invention. They are intended to be illustrative and are not to be construed as limiting in any way.

### EXAMPLES

### Materials

For the experiments described below, Compound A (Mw=418.4 g/mol, melting point (Tm)=125° C., logP=3.998, pKa=5.41), Compound B (Mw=385.4 g/mol, Tm=208° C.), abiraterone acetate (Mw=391.6 g/mol, Tm=147° C., logP=5.12, pKa=5.19), Ibrutinib (Mw=440.5 g/mol, Tm=149-158° C., pKa=3.74), Cannabidiol (BSPG Laboratories, Sandwich, Kent, UK) with a specification 98%-102% pure CBD isolate with impurity profile<0.15% and zero THC with LOD 0.000004% (Brains Bio, BSPG Laboratories, Sandwich, Kent, UK) ((Mw=314 g/mol, Tm=67.5° C., pka=9.7), beta-lactoglobulin with a purity fraction of >98% in the protein fraction (BLG), polyvinylpyrrolidine K25 (PVP K25, Kollidon® 25, BASF, Ludwigshafen, Germany), methacrylic acid-ethyl acrylate copolymer (1:1) Type A (Eudragit® L100-55, Evonik, Darmstadt, Germany), polystyrene sulfonate (PSS, Sigma Aldrich, St. Louis, MO, USA), microcrystalline cellulose (MCC, Avicel PH 102, DuPont Nutrition, Ireland), poloxamer 407 (Kolliphor® P 407, BASF, Ludwigshafen, Germany) were used.

### Methods

### X-Ray Powder Diffraction (XRPD) for Investigation of the Solid State

The solid state of the prepared formulation was investigated using an X′Pert PANanalytical PRO X-ray diffractometer (PANanalytical, Almelo, The Netherlands) with Cu Kα radiation (λ=1.54187 Å). Samples were scanned in reflectance mode from 5 to 30 2θ, with a scan speed of 0.067 2θ/s and a step size of 0.026 2θ. The acceleration voltage and current are 45 KV and 40 mA, respectively.

Modulate Temperature Differential Scanning Calorimetry (mDSC) for Measurement of the Glass Transition Temperature (Tg)

The mDSC thermograms of the samples were collected using a Discovery DSC (TA instruments, New Castle, USA) under a nitrogen gas flow of 50 ml/min. The samples were analysed at a heating rate of 2° C./min with an underlying modulation temperature amplitude of 0.2120° C. and a period of 40 s. For Cannabidiol, the samples were heated from −40° C. to 150° C., for all the other compounds, the samples were heated from 0° C. to 250° C. A total of 4-8 mg sample powder was filled into aluminium Tzero pans and sealed with an aluminium Tzero lid. The glass transition temperature (Tg) was determined as the midpoint from the reversing heat flow signal.

### Example 1-Preparation of Compound a Binary and Ternary Co-Amorphous Forms

### Preparation Method

Binary co-amorphous forms at 25% drug loading were prepared using vibrational ball milling (MixerMill MM400, Retsch GmbH & Co., Haan, Germany) in a 4° C. cold room at 30 Hz. A mass of 125 mg of Compound A and 375 mg of BLG were weighed into a 25 ml milling jar and milling was performed with two 12 mm stainless steel balls for 60 min.

Ternary co-amorphous forms at 25% drug loading were also prepared using vibrational ball milling in a 4° C. cold room at 30 Hz. A mass of 100 mg of Compound A, 150 mg of PVPK25 and 150 mg of BLG were weighed into a 25 ml milling jar and milling was performed with two 12 mm stainless steel balls for 120 min.

### Dissolution Method-Dissolution of Compound a Binary and Ternary Co-Amorphous Forms in FaSSGF and FaSSIF-V2

The powder dissolution of Compound A binary and ternary co-amorphous forms was determined at room temperature in fasted state simulated gastric fluid (FaSSGF, Biorelevant) and fasted state simulated intestinal fluid V2 (FaSSIF-V2, Biorelevant) as dissolution media. Samples equivalent to 20 mg of Compound A were added into an Erlenmeyer flask containing 20 ml of dissolution medium under stirring at a speed of 200 rpm.

A volume of 2 ml of dissolution medium was withdrawn from the dissolution vessels at 5, 10, 20, 40 60, 90 and 120 min, and immediately replaced by 2 ml of fresh dissolution medium. The dissolution samples were filtered through a 0.45 μm filter and diluted using acetonitrile, and subsequently filtered again through a 0.45 μm filter. Finally, the samples were analyzed toward drug content using high performance liquid chromatography (HPLC). The dissolution profile was plotted as cumulative concentration.

### Results

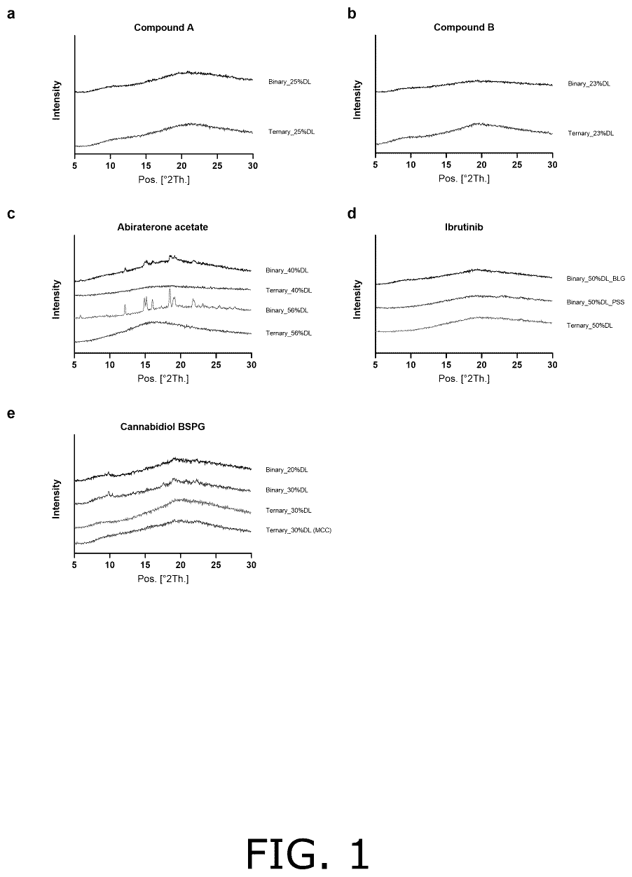

The appearance of the amorphous halo (FIG. 1a) showed the success in amorphization both for the binary and ternary co-amorphous forms. The appearance of a single Tg in the mDSC thermograms of the ternary co-amorphous form (Table 1) suggested that it is a homogeneous single phase co-amorphous system.

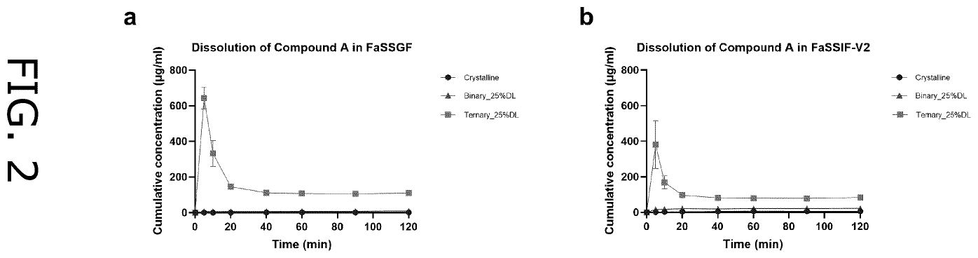

As shown in FIG. 2, in FaSSGF, the ternary co-amorphous form releases initially approx. 650 μg/ml of Compound A (5 min) followed by a precipitation of Compound A to concentration levels of approx. 110 μg/ml. In comparison, the binary co-amorphous forms only reached low concentrations of approx. 6-10 μg/ml during the whole dissolution period.

In FaSSIF-V2, the ternary co-amorphous form releases initially approx. 380 μg/ml of Compound A (5 min) followed by a precipitation of Compound A to concentration levels of approx. 83 μg/ml. In comparison, the binary co-amorphous forms again only reached low concentrations of 15-25 μg/ml during the whole dissolution period. These results demonstrated that the ternary co-amorphous form formulated with BLG and PVPK25 increased the dissolution rate and solubility of Compound A significantly compared to the binary co-amorphous forms.

### Example 2-Preparation of Compound B Binary and Ternary Co-Amorphous Forms

### Preparation Method

Binary and ternary co-amorphous forms at 23% drug loading were prepared by spray drying using a ProCepT spray dryer (ProCepT, Zelzate, Belgium), equipped with an extended column and large cyclone. For the binary co-amorphous form, Compound B and BLG were dissolved in formic acid at a solid concentration of 5%. For the ternary co-amorphous form, Compound B, BLG and Eudragit L100-55 (Compound B: BLG: EudL ratio: 23:54:23, w/w/w) were dissolved in formic acid at a solid concentration of 6.5%. The spray drying process was conducted under the following process settings: inlet temperature of 110° C., inlet gas flow of 0.4 m3/min, cyclone gas flow of 300 l/min, nozzle gas flow of 6 l/min and feed rate of approx. 2.5 g/min. The column out temperature was recorded to be approx. 67° C.

### Dissolution Method-Dissolution of Compound B Binary and Ternary Co-Amorphous Forms in FaSSIF-V1

The powder dissolution of Compound B binary and ternary co-amorphous forms was determined at room temperature in fasted state simulated intestinal fluid V1 (FaSSIF-V1, Biorelevant) as dissolution medium. Samples equivalent to 10 mg of Compound B were added into an Erlenmeyer flask containing 10 ml of dissolution medium under stirring at speed of 200 rpm. A volume of 1 ml of dissolution medium was withdrawn from the dissolution vessels at 5, 10, 20, 40, 80 and 120 min. The dissolution samples were filtered through a 0.22 μm filter and diluted using acetonitrile, and subsequently filtered again through a 0.22 μm filter. Finally, the samples were analyzed toward drug content using HPLC. The dissolution profile was demonstrated as concentration at each time point.

### Results

The appearance of the amorphous halo (FIG. 1b) showed the success in amorphization both for the binary and ternary co-amorphous forms. The appearance of two Tg's in the mDSC thermograms of the ternary co-amorphous form (Table 1) suggested that the ternary co-amorphous form resulted in two phase co-amorphous system.

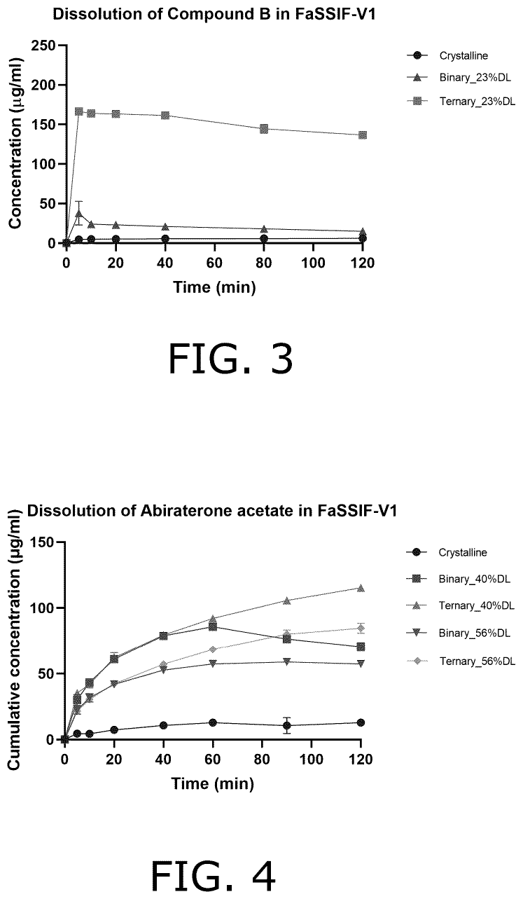

As shown in FIG. 3, the ternary co-amorphous form of Compound B released initially approx. 166 g/ml of Compound B (5 min) followed by a slight decrease in concentrations to approx. 136 μg/ml after 120 min. In comparison, the binary co-amorphous form of Compound B released initially approx. 38 μg/ml of Compound B (5 min) followed by a pronounced precipitation of Compound B to concentration levels of approx. 15 μg/ml. This study demonstrated that a ternary co-amorphous form formulated with BLG and Eudragit L100-55 increased solubility and prevented precipitation of Compound B.

### Example 3-Preparation of Abiraterone Acetate Binary and Ternary Co-Amorphous Forms

### Preparation Method

Binary and ternary co-amorphous forms at 40% (w/w) and 56% (w/w) drug loadings were prepared by spray drying using a ProCepT spray dryer, equipped with an extended column and large cyclone. For the binary formulation at 40% (w/w) drug loading, Abiraterone acetate and BLG were dissolved in a 50% (v/v) aqueous 1-propanol at a solid concentration of 4%. For the binary co-amorphous form at 56% (w/w) drug loading, Abiraterone acetate and BLG were dissolved in a 57% (v/v) aqueous 1-propanol at a solid concentration of 3.5%. For ternary co-amorphous forms at 40% (w/w) and 56% (w/w) drug loadings, Abiraterone acetate together with BLG and Eudragit L100-55 at a weight ratio of 1:1 (BLG: Eudragit L100-55) were dissolved in a mixture of 1-propanol, water and formic acid (10:10:2, v/v/v) at a solid concentration of 4.5%. The spray drying process was conducted under the following process settings: inlet temperature of 100° C., inlet gas flow of 0.4 m3/min, cyclone gas flow of 300 l/min, nozzle gas flow of 6 l/min and feed rate of approx. 4 g/min. The column out temperature was recorded to be approx. 58° C.

### Dissolution Method-Dissolution of Abiraterone Acetate Binary and Ternary Co-Amorphous Forms in in FaSSIF-V1

The powder dissolution of Abiraterone acetate binary and ternary co-amorphous forms was determined at room temperature in fasted state simulated intestinal fluid V1 (FaSSIF-V1, Biorelevant) as dissolution media. Samples equivalent to 20 mg of Abiraterone acetate were added into an Erlenmeyer flask containing 20 ml of dissolution medium under stirring at a speed of 200 rpm.

A volume of 2 ml of dissolution medium was withdrawn from the dissolution vessels at 5, 10, 20, 40 60, 90 and 120 min, and immediately replaced by 2 ml of fresh dissolution medium. The dissolution samples were filtered through a 0.45 μm filter and diluted using acetonitrile, and subsequently filtered again through a 0.45 μm filter. Finally, the samples were analyzed towards drug content using high performance liquid chromatography (HPLC). The dissolution profile was plotted as cumulative concentration.

### Results

The appearance of the amorphous halo (FIG. 1c) showed the success in amorphization for the ternary co-amorphous forms. The appearance of a single Tg in the mDSC thermograms of the ternary formulations (Table 1) suggested that both formulations resulted in homogeneous single phase co-amorphous systems. However, the binary co-amorphous forms showed remaining crystalline diffractions in the diffractograms indicating that they were not fully amorphous.

As shown in FIG. 4, the ternary co-amorphous form at 40% drug loading released approx. 115 μg/ml of Abiraterone acetate (120 min), the ternary co-amorphous form at 56% drug loading released approx. 85 μg/ml of Abiraterone acetate (120 min). In comparison, the binary formulation at 40% drug loading released approx. 85 μg/ml of Abiraterone acetate (60 min), followed by a precipitation of Abiraterone acetate to concentration levels of approx. 70 μg/ml after 120 min. The binary formulation at 56% drug loading only released approx. 57 μg/ml of Abiraterone acetate (120 min). These results demonstrated that the ternary co-amorphous forms with BLG and Eudragit L 100-55 increased the dissolution rate and solubility of Abiraterone acetate significantly compared to the binary formulations.

### Example 4-Preparation of Ibrutinib Binary and Ternary Co-Amorphous Forms

### Preparation Method

Binary and ternary co-amorphous forms at 50% drug loading were prepared by spray drying using a ProCepT spray dryer (ProCepT, Zelzate, Belgium), equipped with an extended column and a large cyclone. For the binary co-amorphous forms, Ibrutinib together with BLG or polystyrene sulfonate (PSS) were dissolved in a mixture of methanol and water (74:26, v/v) at a solid concentration of 2.6%. For the ternary co-amorphous form, Ibrutinib together with BLG and PSS at a weight ratio of 1:1 (BLG: PSS) were dissolved in a mixture of methanol and water (74:26, v/v) at a solid concentration of 2.6%. The spray drying process was conducted under the following process settings: inlet temperature of 120° C., inlet gas flow of 0.4 m3/min, cyclone gas flow of 300 l/min, nozzle gas flow of 6 l/min and feed rate of approx. 4 g/min. The column out temperature was recorded to be approx. 64° C.

### Dissolution Method-Dissolution of Ibrutinib Binary and Ternary Co-Amorphous Forms in FaSSIF-V2

The powder dissolution of Ibrutinib binary and ternary co-amorphous forms was determined at room temperature in fasted state simulated intestinal fluid V2 (FaSSIF-V2, Biorelevant) as dissolution medium. Samples equivalent to 20 mg of Ibrutinib were added into an Erlenmeyer flask containing 20 ml of dissolution medium under stirring at speed of 250 rpm. A volume of 2 ml of dissolution medium was withdrawn from the dissolution vessels at 5, 10, 20, 40, 60, 90 and 120 min. The dissolution samples were centrifuged for 3 min at 14600 rpm, the supernatant was collected and diluted using acetonitrile. Subsequently the samples were filtered through a 0.45 μm filter and analyzed toward drug content using HPLC. The dissolution profile was demonstrated as concentration at each time point.

### Results

The appearance of the amorphous halo (FIG. 1d) showed the success in amorphization both for the binary and ternary co-amorphous forms. The appearance of a single Tg in the mDSC thermograms of the ternary formulation (Table 1) suggested that the formulation resulted in homogeneous single phase co-amorphous systems.

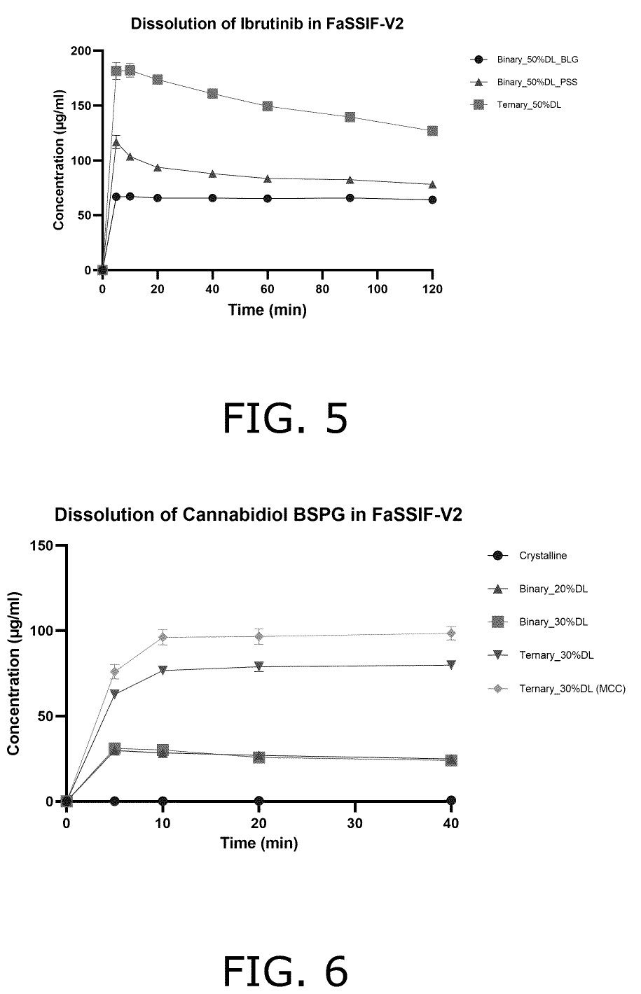

As shown in FIG. 5, the ternary co-amorphous form of Ibrutinib released initially approx. 181 μg/ml of Ibrutinib (5 min) followed by a decrease in concentrations to approx. 127 μg/ml after 120 min. In comparison, the binary co-amorphous form with PSS only released approx. 117 μg/ml of Ibrutinib (5 min) followed by a decrease in concentrations to approx. 80 μg/ml (120 min), and the binary co-amorphous form with BLG only released 67 μg/ml during the whole dissolution period. This study demonstrated that a ternary co-amorphous form formulated with BLG and PSS increased solubility of Ibrutinib compared to the binary formulations.

### Example 5-Preparation of Cannabidiol Binary and Ternary Co-Amorphous Forms

### Preparation Method

Binary and ternary co-amorphous forms were prepared by spray drying using a ProCepT spray dryer (ProCepT, Zelzate, Belgium), equipped with an extended column and a large cyclone. Cannabidiol and BLG together with or without poloxamer 407 were dissolved in a mixture of methanol and water at a solid concentration of 3-4%. Additionally, a ternary co-amorphous form with insoluble microcrystalline cellulose (MCC) was spray dried from the same solution as the ternary formulation but by additionally dispersing MCC in the solution containing cannabidiol, BLG and poloxamer 407. The binary formulations with BLG were prepared at drug loadings 20 and 30%. The ternary formulation was prepared at 30% drug loading and is abbreviated as Cannabidiol_30% DL. For the ternary formulation with additional MCC, the ratio between Cannabidiol BSPG, BLG and Poloxamer 407 was 30:55:15, however, with the dispersed MCC, the ratio between the components was 23:42:12:23. Since the ratio for the ternary formulation remains the same, this formulation is abbreviated Cannabidiol_30% DL (MCC). The spray drying process was conducted under the following process settings: inlet temperature of 100° C., inlet gas flow of 0.4 m3/min, cyclone gas flow of 300 l/min, nozzle gas flow of 6 l/min and feed rate of approx. 4 g/min.

### Dissolution Method-Dissolution of Cannabidiol BSPG Binary and Ternary Co-Amorphous Forms in FaSSIF-V2

The powder dissolution of Cannabidiol binary and ternary co-amorphous forms was determined at 37° C. in fasted state simulated intestinal fluid V2 (FaSSIF-V2, Biorelevant) as dissolution medium. Samples equivalent to 10 mg of Cannabidiol were added into a USP II setup on an ERWEKA DT 70 dissolution tester (ERWEKA, Langen, Germany) containing 100 ml of dissolution medium under a mini paddle stirring at speed of 100 rpm. A volume of 2 ml of dissolution medium was withdrawn from the dissolution vessels at 5, 10, 20 and 40 min. The dissolution samples were centrifuged for 3 min at 14500 rpm, the supernatant was collected and diluted using methanol. Subsequently the samples were filtered through a 0.45 μm filter and analyzed toward drug content using HPLC. The dissolution profile was demonstrated as concentration at each time point.

### Results

The appearance of the amorphous halo (FIG. 1e) showed the success in amorphization for the ternary co-amorphous forms. The appearance of a single Tg in the mDSC thermograms of the ternary formulations (Table 1) suggested that both formulations resulted in homogeneous single phase co-amorphous systems. However, the binary co-amorphous forms at 20% and 30% drug loadings showed remaining crystalline diffractions in the diffractograms indicating that they were not fully amorphous.

As shown in FIG. 6, the ternary co-amorphous forms of Cannabidiol at 30% drug loading with and without MCC released approx. 98 μg/ml and 80 μg/ml of Cannabidiol (40 min), respectively. In comparison, the binary co-amorphous forms at 30% drug loading and even lower drug loading (20%) only released approx. 31 μg/ml of Cannabidiol (5 min) followed by a decrease in concentrations to approx. 24 μg/ml (40 min). This study demonstrated that a ternary co-amorphous form formulated with BLG and poloxamer 407 increased solubility of Cannabidiol compared to the binary formulations.

### Example 6-Preparation of Olaparib Binary and Ternary Co-Amorphous Forms

A ternary Olaparib formulation comprising Olaparib, BLG and Eudragit L in the weight ratio of 60:25:15 can be prepared by dissolving Olaparib, BLG and Eudragit L in a mixture of ethanol and water, or a mixture of ethanol, water and formic acid, the resultant solution was then spray dried as described previously producing a ternary co-amorphous form of Olaparib in BLG and Eudragit L.

TABLE 1
mDSC data on the Tg of the ternary co-amorphous forms.

| | Ternary co-amorphous |
|---|---|
| Tg (° C.) | forms |
| Compound A_25% DL | 143.0 |
| Compound B_23% DL | 99.2 & 160.1 |
| Abiraterone acetate_40% DL | 90.3 |
| Abiraterone acetate_56% DL | 53.2 |
| Ibrutinib_50% DL | 60.0 |
| Cannabidiol_30% DL | −16.7 * |
| Cannabidiol_30% DL (MCC) | −17.7 * |

* Samples were only heated to 150° C., at which temperature the co-amorphous form began to decompose.

1. A co-amorphous form of a drug, a nutraceutical, or a dietary supplement with a protein and a water-soluble polymer, wherein the protein is beta-lactoglobulin.

2-10. (canceled)

11. The co-amorphous form according to claim 1, wherein the purity of the beta-lactoglobulin is at least 92% (w/w) of the total amount of protein of the co-amorphous form.

12. The co-amorphous form according to claim 1, wherein the water-soluble polymer is selected from HPMCAS, PVPVA, PVP, PVAc, PVOH, PVAP (Polyvinyl acetate phthalate), Poloxamer type polymers, Poloxamer 188, PEG, MC, HPMC, HPC, HEC, HPMCAS, HPMCP, chitosan, polymethacrylate-based copolymers, Eudragit type polymers, polyacrylic acid polymers, Carbomer, Carbopol 940, carboxymethyl cellulose acetate butyrate, a cellulose derivative, modified starches, polysaccharides, polyvinyl caprolactam-polyvinyl acetate-polyethylene glycol graft co-polymer, Soluplus, polystyrene sulfonate (PSS), or any mixture thereof.

13. The co-amorphous form according to claim 12, wherein the water-soluble polymer is selected from polyvinyl pyrrolidone, Poloxamer type polymers, polymethacrylate-based copolymers, polystyrene sulfonate, or any mixture thereof.

14. The co-amorphous form according to claim 1, wherein the drug, nutraceutical or dietary supplement comprises a drug compound.

15. The co-amorphous form according to claim 14, wherein the drug compound is classified in BCS class II or IV.

16. A method of preparing the co-amorphous form according to claim 1, wherein the drug, nutraceutical, or dietary supplement, protein, and water-soluble polymer together are subjected to spray drying, solvent evaporation, freeze drying, precipitation from supercritical fluids, melt quenching, hot melt extrusion, electrospinning, 2D printing, 3D printing, a milling process, wet milling, ball milling or cryo-milling.

17. A method for preparing a protein and a water-soluble polymer in a co-amorphous form with a drug, nutraceutical, or dietary supplement, comprising incorporating beta-lactoglobulin, a water-soluble polymer, and a drug, nutraceutical, or dietary supplement in a co-amorphous form.

18. A co-amorphous form of a drug, a nutraceutical, or a dietary supplement with a protein and a water-soluble polymer, wherein the water-soluble polymer is selected from polyvinyl pyrrolidone, Poloxamer type polymers, polymethacrylate-based copolymers, polystyrene sulfonate, or any mixture thereof.

19. The co-amorphous form according to claim 18, wherein the water-soluble polymer is polystyrene sulfonate.
